(19) United States
(12) Patent Application Publication
(10) Pub. No.: US 2008/0267914 A1
(43) Pub. Date: Oct. 30, 2008

(54) Chemical Compounds

(76) Inventors: Michael Christopher Barnes, London (GB); Surinder Singh Chana, London (GB); Helena Dennison, London (GB); Lyn Jennens, London (GB); Neil Mathews, London (GB); Christopher John Pilkington, London (GB); Keith Charles Spencer, London (GB); Alexander James Floyd Thomas, London (GB)

Correspondence Address: ASTRAZENECA R&D BOSTON, 35 GATEHOUSE DRIVE, WALTHAM, MA, 02451-1215, US

(21) Appl. No.: 12/089,301

(22) PCT Filed: Oct. 9, 2006
(86) PCT No.: PCT/GB06/03746

§ 371 Date: Apr. 4, 2008

(30) Foreign Application Priority Data
Oct. 7, 2005 (GB) 0520475.5

(51) Int. Cl. C07D 413/12; C07D 413/14; A61K 31/5377; A61K 31/7056; A61K 38/21; A61P 31/12

(52) U.S. Cl. 424/85.4; 544/119; 514/232.5; 514/234.5; 544/80; 514/43

(57) ABSTRACT

Compounds of formula (Ia) are found to be active in inhibiting replication of flaviviridae viruses (Ia), wherein R₁ and R₂ are the same or different and represent hydrogen, halogen, -L-O—R₃, -L-O-L-A or -L-O-L′-A′, wherein each L is the same or different and represents a direct bond or a C₁-C₄ alkylene group; L′ represents a direct bond or a C₂-C₄ alkylene group; R₃ represents hydrogen, C₁-C₄ alkyl or C₁-C₄ haloalkyl; A represents a 5- to 10-membered heterocyclyl group; and A′ represents a C₆-C₁ aryl group; wherein at least one of R₁ and R₂ is -L-O—R₃, -L-O-L-A or -L-O-L′-A′.

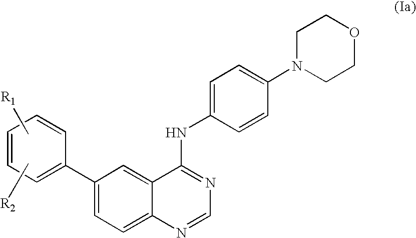

The present invention relates to a series of quinazoline derivatives which are useful in treating or preventing a flaviviridae infection.

Viruses of the family flaviviridae are small, icosahedral, enveloped viruses that contain a positive-sense RNA genome. The family consists of three genera, flavivirus, pestivirus and hepacivirus.

Many of the flaviviridae viruses are important human pathogens. Indeed, the hepacivirus genus includes the hepatitis C virus. However, there exists, as yet, no effective and safe treatment for flaviviridae infections.

WO 98/02434 discloses quinazolines as protein tyrosine kinase inhibitors. None of the compounds specifically disclosed in that document carry a morpholino-aniline-group at the 6-position.

It has now surprisingly been found that the quinazoline derivatives of the formula (Ia) are active in inhibiting replication of flaviviridae viruses and are therefore effective in treating or preventing a flaviviridae infection. These compounds also have particularly beneficial bioavailability. The present invention therefore provides a quinazoline derivative of formula (Ia), or a pharmaceutically acceptable salt thereof,

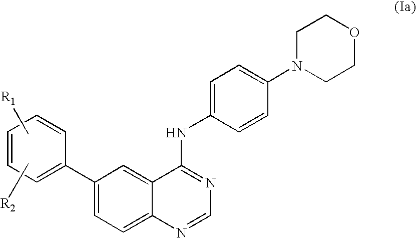

wherein R₁ and R₂ are the same or different and represent hydrogen, halogen, -L-O—R₃, -L-O-L-A or -L-O-L′-A′, wherein
each L is the same or different and represents a direct bond or a C₁-C₄ alkylene group;
L′ represents a direct bond or a C₂-C₄ alkylene group;
R³ represents hydrogen, C₁-C₄ alkyl or C₁-C₄ haloalkyl;
A represents a 5- to 10-membered heterocyclyl group; and
A′ represents a C₆-C₁₀ aryl group;
wherein at least one of R₁ and R₂ is -L-O—R₃, -L-O-L-A or -L-O-L′-A′.

In one embodiment, the quinazoline derivative of formula (Ia) is a quinazoline derivative of formula (I),

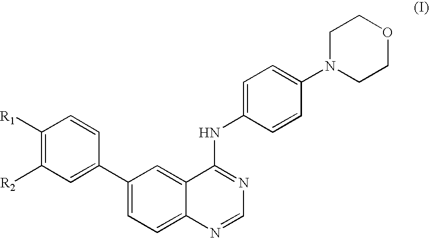

wherein R₁ and R₂ are the same or different and represent hydrogen, halogen, —O—R₃ or —O-L-A, wherein
L represents a C₁-C₄ alkylene group;
R₃ represents hydrogen, C₁-C₂ alkyl or C₁-C₂ haloalkyl; and
A represents a morpholinyl group,
wherein at least one of R₁ and R₂ represents —O—R₃ or —O-L-A.

Typically, R₁ represents —O—R₃ or —O-L-A and R₂ represents hydrogen, halogen, —O—R₃ or —O-L-A.

As used herein, a C₁-C₄ alkyl group or moiety is a linear or branched alkyl group or moiety containing from 1 to 4 carbon atoms. A C₁-C₄ alkyl group or moiety is preferably a C₁-C₂ alkyl group or moiety. Examples of C₁-C₄ alkyl groups and moieties include methyl, ethyl, n-propyl, i-propyl, n-butyl, i-butyl and t-butyl. Examples of C₁-C₂ alkyl groups and moieties include methyl and ethyl.

As used herein, a C₁-C₄ alkylene group or moiety is a linear or branched alkylene group or moiety. Examples include methylene, ethylene and n-propylene groups and moieties, in particular ethylene and n-propylene groups and moieties. A C₂-C₄ alkylene group or moiety is a linear or branched alkylene group or moiety. Examples include ethylene and n-propylene groups and moieties. For the avoidance of doubt, where two alkylene moieties are present in a group, the alkylene moieties may be the same or different.

Typically, as used herein, a C₆-C₁₀ aryl group or moiety is phenyl or naphthyl. Phenyl is preferred.

As used herein, a halogen is typically chlorine, fluorine, bromine or iodine and is preferably chlorine, bromine or fluorine, in particular fluorine.

As used herein a haloalkyl group is typically a said alkyl group substituted by one or more said halogen atoms. Typically, it is substituted by 1, 2 or 3 said halogen atoms, particularly by 1, 2 or 3 fluorine atoms. Preferred haloalkyl groups include —CF₃ and —CHF₂.

As used herein, a 5- to 10-membered heterocyclyl group or moiety is a monocyclic non-aromatic, saturated or unsaturated C₅-C₁₀ carbocyclic ring in which one or more, for example 1, 2 or 3, of the carbon atoms are replaced with a moiety selected from N, O, S, S(O) and S(O)₂, for example N and/or O. Typically, it is a 5- to 6-membered ring. Typically, it a saturated ring.

Suitable heterocyclyl groups and moieties include pyrazolidinyl, piperidyl, piperazinyl, thiomorpholinyl, morpholinyl, pyrrolidinyl, 1,3-dioxolanyl and 1,4-dioxolyl groups and moieties. Morpholinyl is particularly preferred.

Typically, the aryl and heterocyclyl moieties in the R₁ and R₂ substituents are unsubstituted.

In the compounds of formula (Ia), R₁ and R₂ are typically located at the 3 and 4 positions of the phenyl ring, in other words they are typically meta and para relative to the quinazoline ring. R₁ is preferably in position 4 (para) and R₂ is preferably in position 3 (meta).

Typically, one or none of R₁ and R₂ represents -L-O-L-A or -L-O-L′-A′. Thus, when R₁ represents hydrogen, halogen or -L-O—R₃, R₂ typically represents hydrogen, halogen, -L-O—R₃, -L-O-L-A or -L-O-L′-A′, provided that one of R₁ and R₂ is -L-O—R₃, -L-O-L-A or -L-O-L′-A′. However, when R₁ represents -L-O-L-A or -L-O-L′-A′, R₂ typically represents hydrogen, halogen or -L-O—R₃.

Typically, R₁ represents -L-O—R₃, -L-O-L-A or -L-O-L′-A′, preferably -L-O—R₃ or -L-O-L-A. Typically, R₂ represents hydrogen, halogen -L-O—R₃, -L-O-L-A or -L-O-L′-A′, preferably halogen -L-O—R₃, -L-O-L-A or -L-O-L′-A′, more preferably halogen, -L-O—R₃ or -L-O-L-A. Preferably, when R₁ represents -L-O—R₃, R₂ represents hydrogen, halogen -L-O—R₃, -L-O-L-A or -L-O-L′-A′, preferably halogen -L-O—R₃, -L-O-L-A or -L-O-L′-A′, more preferably halogen, -L-O—R₃ or -L-O-L-A. Alternatively, when R₁ represents -L-O-L-A or L-O-L′-A′, R₂ preferably represents hydrogen, halogen or -L-O—R₃, preferably halogen or -L-O—R₃.

When R₁ or R₂ represents -L-O—R₃, the group L is typically a direct bond or C₁-C₂ alkylene, preferably a direct bond. R₃ is typically hydrogen, C₁-C₂ alkyl or C₁-C₂ haloalkyl. -L-O—R₃ therefore typically represents —O—R₃ wherein R₃ is hydrogen, C₁-C₂ alkyl or C₁-C₂ haloalkyl.

When R₁ or R₂ represents -L-O-L-A, it is typically a group —O-L-A or —(C₁-C₂ alkylene)-O-L-A, preferably a group —O-L-A, wherein L is a direct bond or a C₁-C₄ alkylene group, preferably a C₁-C₄ alkylene group. A is typically a morpholinyl group.

When R₁ or R₂ represents -L-O-L′-A′, it is typically a group —O-L′-A′ or —(C₁-C₂ alkylene)-O-L′-A′, preferably a group —O-L′-A′, wherein L′ is a direct bond or a C₂-C₄ alkylene group, preferably a C₂-C₄ alkylene group. A′ is typically a phenyl group.

In a preferred embodiment of the invention, the quinazoline derivative of formula (Ia) is a quinazoline derivative of formula (I) wherein R₁ and R₂ are the same or different and represent hydrogen, halogen, —O—R₃, —(C₁-C₂ alkylene)-O—R₃, —O-L-A, —(C₁-C₂ alkylene)-O-L-A, —O-L′-A′ or —(C₁-C₂ alkylene)-O-L′-A′, wherein

L represents a direct bond or a C₁-C₄ alkylene group;
L′ represents a direct bond or a C₂-C₄ alkylene group;
R₃ represents hydrogen, C₁-C₄ alkyl or C₁-C₄ haloalkyl;
A represents a morpholinyl group; and
A′ represents phenyl;
wherein at least one of R₁ and R₂ represents —O—R₃, —(C₁-C₂ alkylene)-O—R₃, —O-L-A, —(C₁-C₂ alkylene)-O-L-A, —O-L′-A′ or —(C₁-C₂ alkylene)-O-L′-A′.

Typically in this embodiment, R₁ represents —O—R₃, —(C₁-C₂ alkylene)-O—R₃, —O-L-A, —(C₁-C₂ alkylene)-O-L-A, —O-L′-A′ or —(C₁-C₂ alkylene)-O-L′-A′ and R₂ represents hydrogen, halogen, —O—R₃, —(C₁-C₂ alkylene)-O—R₃, —O-L-A, —(C₁-C₂ alkylene)-O-L-A, —O-L′-A′ or —(C₁-C₂ alkylene)-O-L′-A′, with the proviso that when R₁ represents —O-L-A, —(C₁-C₂ alkylene)-O-L-A, —O-L′-A′ or —(C₁-C₂ alkylene)-O-L′-A′, then R₂ represents hydrogen, halogen, —O—R₃ or —(C₁-C₂ alkylene)-O—R₃, preferably halogen, —O—R₃ or —(C₁-C₂ alkylene)-O—R₃.

In a further preferred embodiment of the invention, the quinazoline derivative of formula (Ia) is a quinazoline derivative of formula (I) wherein R₁ and R₂ are the same or different and represent hydrogen, halogen, —O—R₃, —O-L-A or —O-L′-A′, wherein

L represents a C₁-C₄ alkylene group;
L′ represents a C₂-C₄ alkylene group;
R₃ represents hydrogen, C₁-C₂ alkyl or C₁-C₂ haloalkyl;
A represents morpholinyl; and
A′ represents phenyl;
wherein at least one of R₁ and R₂ is —O—R₃, —O-L-A or —O-L′-A′.

In a preferred aspect of this embodiment, R₁ represents —O—R₃, —O-L-A or —O-L′-A′, and R₂ represents hydrogen, halogen, —O—R₃, —O-L-A or —O-L′-A′, preferably halogen, —O—R₃, —O-L-A or —O-L′-A′, provided that when R₁ represents —O-L-A or —O-L′-A′, R₂ represents hydrogen, halogen or —O—R₃, preferably halogen or —O—R₃.

In a further preferred embodiment of the invention, the quinazoline derivative of formula (Ia) is a quinazoline derivative of formula (I) wherein R₁ and R₂ are the same or different and represent hydrogen, halogen, —O—R₃ or —O-L-A, wherein

L represents a C₁-C₄ alkylene group;
R₃ represents hydrogen, C₁-C₂ alkyl or C₁-C₂ haloalkyl; and
A represents morpholinyl.

In a preferred aspect of this embodiment, R₁ represents —O—R₃ or —O-L-A and R₂ represents hydrogen, halogen, —O—R₃ or —O-L-A, preferably halogen, —O—R₃ or —O-L-A, provided that when R₁ represents —O-L-A, R₂ represents hydrogen, halogen or —O—R₃, preferably halogen or —O—R₃.

Particularly preferred compounds of formula (Ia) include:
6-[4-(2-Morpholin-4-yl-ethoxy)-phenyl]-quinazolin-4-yl}-(4-morpholin-4-yl-phenyl)-amine
(4-Morpholin-4-yl-phenyl)-{6-[4-(3-morpholin-4-yl-propoxy)-phenyl]-quinazolin-4-yl}-amine
[6-(3,4-Dimethoxy-phenyl)-quinazolin-4-yl]-(4-morpholin-4-yl-phenyl)-amine
[6-(3,4-Diethoxy-phenyl)-quinazolin-4-yl]-(4-morpholin-4-yl-phenyl)-amine
{6-[3-Fluoro-4-(2-morpholin-4-yl-ethoxy)-phenyl]-quinazoline-4-yl}-(4-morpholin-4-yl-phenyl)-amine
2-Methoxy-5-[4-(4-morpholin-4-yl-phenylamino)-quinazolin-6-yl]-phenol
[6-(3,4-Bis-trifluoromethoxy-phenyl)-quinazolin-4-yl]-(4-morpholin-4-yl-phenyl)-amine
[6-(3,4-Bis-difluoromethoxy-phenyl)-quinazolin-4-yl]-(4-morpholin-4-yl-phenyl)-amine
{6-[4-Methoxy-3-(2-morpholin-4-yl-ethoxy)-phenyl]-quinazolin-4-yl}-(4-morpholin-4-yl-phenyl)-amine
{6-[3-Methoxy-4-(2-morpholin-4-yl-ethoxy)-phenyl]-quinazolin-4-yl}-(4-morpholin-4-yl-phenyl)-amine
{6-[3-Fluoro-4-(3-morpholin-4-yl-propoxy)-phenyl]-quinazolin-4-yl}-(4-morpholin-4-yl-phenyl)-amine
[6-(3-Ethoxy-4-methoxy-phenyl)-quinazolin-4-yl]-(4-morpholin-4-yl-phenyl)-amine
[6-(4-Ethoxy-3-methoxy-phenyl)-quinazolin-4-yl]-(4-morpholin-4-yl-phenyl)-amine and pharmaceutically acceptable salts thereof.

Compounds of formula (Ia) containing one or more chiral centre may be used in enantiomerically or diastereoisomerically pure form, or in the form of a mixture of isomers. For the avoidance of doubt, the compounds of formula (Ia) can, if desired, be used in the form of solvates. Further, for the avoidance of doubt, the compounds of the invention may be used in any tautomeric form.

As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases such as alkyl amines, aralkyl amines and heterocyclic amines.

The compounds of the invention in which R1 is other than hydrogen or halogen, can, for example, be prepared according to the following reaction schemes.

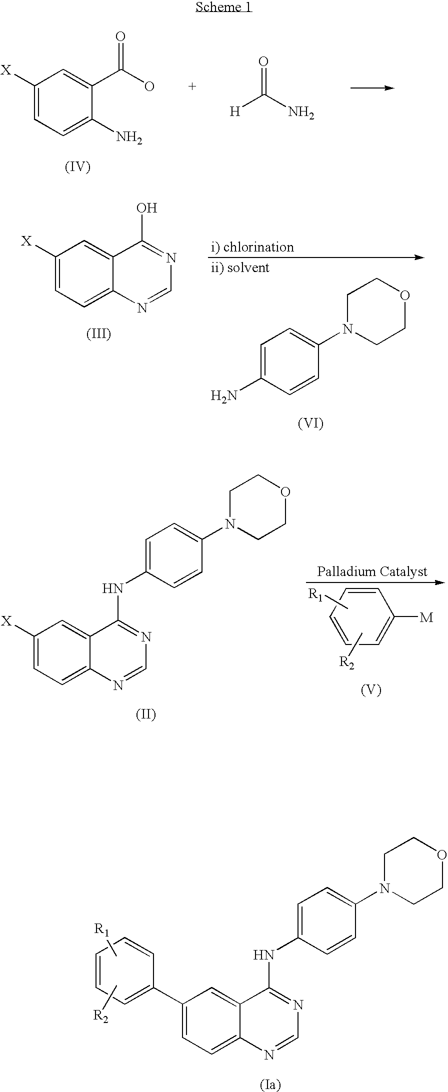

As will be evident to one of skill in the art, X in the above reaction schemes is an appropriate leaving group, for example halogen.

Referring to Scheme 1, the treatment of compounds of formula (II) with an organometallic reagent (V) is conveniently carried out in a suitable solvent (such as tetrahydrofuran, dimethylformamide or toluene) and at elevated temperature (eg from 50° C. to reflux). Conveniently, the reaction is performed under palladium catalysis (eg 20 mol % tris (dibenzylideneacetone)dipalladium (II) or 20 mol % dichlorobis (triphenylphosphine)palladium (0)) in the presence of an organic base (eg triethylamine) or an inorganic base (eg sodium carbonate or potassium phosphate). Where reagent (V) is an organostannane (eg M=SnBu₃), one skilled in the art will recognise the reaction as an example of a Stille coupling where additional additives may be beneficial eg lithium chloride, silver oxide and conveniently the reaction is performed in toluene and at reflux temperature. Where reagent (V) is a boronic acid derivative, one skilled in the art will recognise the reaction as an example of a Suzuki-Miyaura coupling which may be conveniently performed at 60° C. in tetrahydrofuran.

Referring to Scheme 1, the conversion of compounds of formula (III) to compounds of formula (II) is accomplished by converting the 4-hydroxy group of compounds of formula (III) to a suitable leaving group eg chloro using a reagent such as thionyl chloride as solvent with the addition of a catalytic activator eg dimethylformamide, and subsequent reaction with 4-morpholinoaniline in a suitable solvent eg acetonitrile.

Referring to Scheme 1, the conversion of compounds of formula (IV) to compounds of formula (III) will be well known to one skilled in the art, being conveniently performed with formamide as solvent and at elevated temperature eg reflux.

Compounds of formula (Ia) in which R₁ or R₂ is a group —OR₃, —O-L-A or —O-L′-A′ can alternatively be produced by the reaction shown in Scheme 2 below. The reaction is typically carried out in the presence of acetic acid at a temperature of about 120° C. and for a period of about 1 hour.

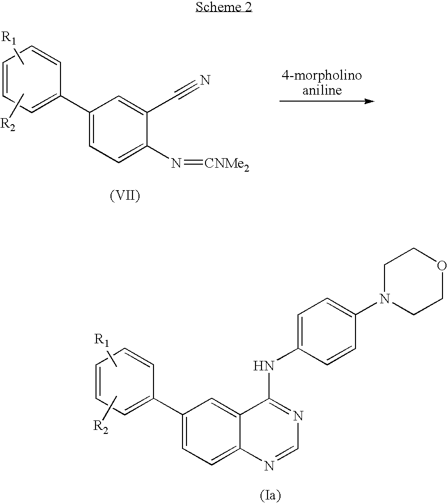

The compounds of formula (VII) used as a starting material in Scheme 2 can be prepared by one of the reactions depicted in Scheme 3 below. In the Schemes 2 and 3, the groups R₁ and R₂ may represent protecting groups, such as benzyl, which can be replaced by the desired R₁ or R₂ group by methods known in the art following reaction. Deprotection can be carried out before or after conversion of the compound of formula (VII) to the compound of formula (Ia).

Referring to scheme 3, each reaction involving an organometallic reagent is conveniently carried out in the same manner as the reaction between the compounds of formulae (II) and (V) described above with reference to Scheme 1. The organometallic compounds each typically have a group M which is B(OR′)₂ or SnR₃, preferably B(OR′)₂. The coupling reactions are thus typically Suzuki-Miyaura or Stille coupling reactions as described above. As the skilled person in the art will appreciate, the group X in the compounds depicted in Scheme 3 is an appropriate leaving group such as I or Br, preferably I.

Referring to Scheme 3, the compound of formula (VIIIa) can be converted to a compound of formula (VIIIc) by reaction with dimethyl formamide dimethylacetal at about 100° C. for approximately 1.5 hours. Similarly, compounds of formula (VIIa) can be converted to compounds of formula (VII) by the same reaction.

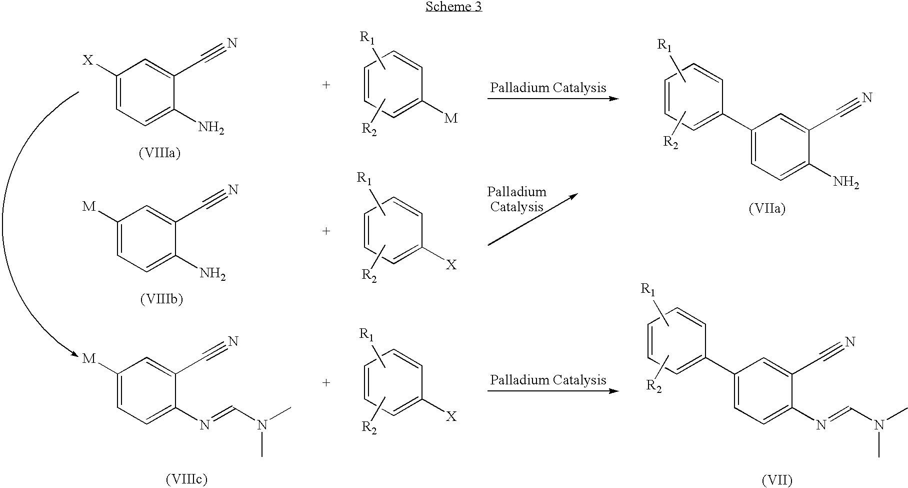

The starting materials in the above reaction schemes are known compounds or can be prepared by analogy with known methods.

The compounds of the present invention are therapeutically useful. The present invention therefore provides a quinazoline derivative of the formula (Ia), as defined above, or a pharmaceutically acceptable salt thereof, for use in treating the human or animal body. Also provided is a pharmaceutical composition comprising a quinazoline derivative of the formula (Ia), as defined above, or a pharmaceutically acceptable salt thereof, and a pharmaceutically acceptable carrier or diluent.

Said pharmaceutical composition typically contains up to 85 wt % of a compound of the invention. More typically, it contains up to 50 wt % of a compound of the invention. Preferred pharmaceutical compositions are sterile and pyrogen free. Further, the pharmaceutical compositions provided by the invention typically contain a compound of the invention which is a substantially pure optical isomer.

As explained above, the compounds of the invention are active against a flaviviridae infection. The present invention therefore provides the use of a quinazoline derivative of the formula (Ia), as defined above, or a pharmaceutically acceptable salt thereof, in the manufacture of a medicament for use in treating or preventing a flaviviridae infection. Also provided is a method for treating a patient suffering from or susceptible to a flaviviridae infection, which method comprises administering to said patient an effective amount of a quinazoline derivative of formula (Ia) or a pharmaceutically acceptable salt thereof.

The flaviviridae family contains three genera. These are hepacivirus, flavivirus and pestivirus. The compounds of the invention are active in treating or preventing a hepacivirus infection, a flavivirus infection or a pestivirus infection.

Typical pestivirus infections which can be treated with the compounds of the invention include bovine viral diarrhea virus, classical swine fever virus and border disease virus.

Typical flavivirus infections which can be treated with the compounds of the invention include yellow fever virus, dengue fever virus, Japanese encephalitis virus and tick borne encephalitis virus.

Typical hepacivirus infections that can be treated with the compounds of the invention include hepatitis C virus.

Compounds of the present invention are especially active against hepatitis C. Typically, said flavivirus is therefore hepatitis C virus.

The compounds of the invention may be administered in a variety of dosage forms. Thus, they can be administered orally, for example as tablets, troches, lozenges, aqueous or oily suspensions, dispersible powders or granules. The compounds of the invention may also be administered parenterally, whether subcutaneously, intravenously, intramuscularly, intrasternally, transdermally or by infusion techniques. The compounds may also be administered as suppositories.

The compounds of the invention are typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for injection or infusion may contain as carrier, for example, sterile water or preferably they may be in the form of sterile, aqueous, isotonic saline solutions.

Compounds of the present invention may be used in conjunction with known anti-viral agents. Preferred known anti-viral agents in this regard are interferon and ribavirin, and derivatives thereof, which are known for the treatment of hepatitis C (Clinical Microbiology Reviews, January 2000, 67-82). The said medicament therefore typically further comprises interferon or a derivative thereof and/or ribavirin or a derivative thereof. Further, the present invention provides a pharmaceutical composition comprising:
(a) a quinazoline derivative of the formula (Ia), as defined above, or a pharmaceutically acceptable salt thereof;
(b) interferon or a derivative thereof and/or ribavirin or a derivative thereof; and
(c) a pharmaceutically acceptable carrier or diluent.

Also provided is a product comprising:
(a) a quinazoline derivative of the formula (Ia), as defined above, or a pharmaceutically acceptable salt thereof; and
(b) interferon or a derivative thereof and/or ribavirin or a derivative thereof, for separate, simultaneous or sequential use in the treatment of the human or animal body.

A preferred interferon derivative is PEG-interferon. A preferred ribavirin derivative is viramidine.

A therapeutically effective amount of a compound of the invention is administered to a patient. A typical dose is from about 0.01 to 100 mg per kg of body weight, according to the activity of the specific compound, the age, weight and conditions of the subject to be treated, the type and severity of the disease and the frequency and route of administration. Preferably, daily dosage levels are from 0.05 to 16 mg per kg of body weight, more preferably, from 0.05 to 1.25 mg per kg of body weight.

The following Examples illustrate the invention. They do not however, limit the invention in any way. In this regard, it is important to understand that the particular assay used in the Examples section is designed only to provide an indication of anti-viral activity. There are many assays available to determine such activity, and a negative result in any one particular assay is therefore not determinative.

### EXAMPLES

All temperatures are in ° C. Thin layer chromatography (TLC) was carried out on Si 60G coated plastic plates with uv254 indicator (Polygram). All NMR spectra were obtained at 250 MHz in d⁶-DMSO unless stated otherwise.

### LC-MS Conditions

Samples were run on a MicroMass ZMD, using electrospray with simultaneous positive—negative ion detection.

Column: Synergi Hydro-RP, 30×4.6 mm I.D, 4 μm.

Gradient: 95:5 to 5:95 v/v H₂O/CH₃CN+0.05% Formic Acid over 4.0 min, hold 3 min, return to 95:5 v/v H₂O/CH₃CN+0.05% Formic Acid over 0.2 min and hold at 95:5 v/v H₂O/CH₃CN+0.05% Formic Acid over 3 min.

Detection: PDA 250-340 nm.

Flow rate: 1.5 ml/min

### Intermediate 1: 2-amino-5-iodobenzonitrile

Prepared by the method of A. Rosowsky & H. Chen, J. Org. Chem. 2001, 66, 7522-7526

¹H NMR (CDCl₃) δ 7.64 (1H, s), 7.55 (1H, dd, J 8.5, 2.5 Hz), 6.53 (1H, d, J=8.5 Hz), 4.66 (2H, br s)

LC-MS rt 2.42 m/z 243 ES−

### Intermediate 2: N′-(2-Cyano-4-iodo-phenyl)-N,N-dimethyl-formamidine

A solution of 2-amino-5-iodobenzonitrile (50 g, 0.2 mol) in DMF-DMA (2.5 eq, 68 ml) heated to 120° for 2 h. The excess DMF-DMA was removed by concentration in vacuo to leave the title compound as a viscous brown oil (61 g, quant)

¹H NMR (CDCl₃) δ 7.79 (1H, d, J=1.9 Hz), 7.65 (1H, dd, J 1.9, 8.5 Hz), 7.57 (1H, s), 6.70 (1H, d, J=8.2 Hz), 3.08 (6H, s)

LC-MS rt 2.1 M/z 300 ES+

### Intermediate 3: 2-Amino-5-(4,4,5,5-tetramethyl-[1,3,2]dioxaborolan-2-yl)-benzonitrile

A mixture of Pd Cl₂(dppf) (3.35 g), potassium acetate (12.07 g) and bis(pinacolato)boron (12.48 g) in dry DMF (80 ml) was treated with intermediate 1 (10 g) and heated to 80 deg for 4 h. The cooled mixture was partitioned between water (400 ml) and CH₂Cl₂ (400 ml). The aqueous phase was further extracted with CH₂Cl₂ (2×100 ml) and the combined organic phases dried and concentrated in vacuo. The residue was purified by chromatography on Silica gel (90 g, retrieve) with 10-30% ethyl acetate in petrol as eluant. Concentration of fractions containing product and trituration with further petrol gave the desired product as a white solid (6.91 g, 69%)

¹H NMR (CDCl₃) δ 7.87 (1H, s), 7.72 (2H, d, J 8.21), 6.7 (1H, d, J=8.2 Hz), 4.57 (2H, br s), 1.31 (12H, s)

LC-MS rt 2.84 m/z 244 ES+

### Intermediate 4: 3-cyano-4-(N′-N′-dimethylformamidinyl)-phenylboronic acid

To a solution of intermediate 2 (10.9 g, 36.4 mmol) in THF (250 ml) was added triisopropyl borate (2 eq, 16.8 ml) and the mixture cooled to −700. Butyl lithium (3 eq, 69 ml of 1.6M in hexanes) was added dropwise and the resulting dark yellow solution stirred for a further 2 h at −70°. Allowed to warm to rt then quenched by the gradual addition of 2M HCl. The mixture was partially concentrated to remove THF and reduce the aqueous volume and the resulting solid isolated by filtration, washed with diethyl ether to remove butyl impurities and dried to give the title compound as an off-white solid (7.62 g, 96%)

¹H NMR δ 8.66 (1H, s), 8.23 (1H, s), 8.11 (1H, d, J 8.2), 7.62 (1H, d, J=8.2 Hz), 3.31 (6H, d)

LC-MS rt 0.55 m/z 218 ES+

### Intermediate 5: N′-[2-Cyano-4-(4,4,5,5-tetramethyl-[1,3,2]dioxaborolan-2-yl)-phenyl]-N,N-dimethyl-formamidine

A suspension of intermediate 3 (750 mg) in DMF-DMA (1 ml) was heated to 100° under N₂ for 30 mins then cooled to rt. The solvent was removed in vacuo and the residue purified by SPE on silica gel (5 g) with 10% ethyl acetate/petrol as eluant. This gave the title compound as a clear oil which crystallised on standing (915 mg, 100%)

¹H(CDCl₃) 7.98 (1H, s), 7.817 (1H, d, J=8.2 Hz), 7.62 (1H, s), 6.92 (1H, d, J=7.6 Hz), 3.1 (3H, s), 3.07 (3H, s), 1.33 (12H, s)

LC-MS rt 2.73 m/z 300 ES+

### Intermediate 6: 4-[3-(4-Iodo-phenoxy)-propyl]-morpholine

A mixture of 4-iodophenol (10 g, 45 mmol) K₂CO₃ (powdered, 4.5 eq) and 1-bromo-3-chloropropane (1.66 eq, 7.5 ml) in MeCN (200 ml) was heated at reflux for 2 h. Concentrated and subjected to an aqueous workup to give a pale oil (14 g). A portion of this oil (3 g) and morpholine (3 eq, 2.64 ml) in DMA (20 ml) was heated to 90° for 72 h. On cooling, the solvent was removed in vacuo and the residue partitioned between EtOAc (100 ml) and sodium carbonate (aq) (50 ml). The organic phase was dried and concentrated to a pale gum that solidified (3.21 g, 91%)

¹H(CDCl₃) 7.53 (2H, d, J=9.5 Hz), 6.66 (2H, d, J=8.5 Hz), 3.98 (2H, t, J=6.3 Hz), 3.7 (4H, m), 2.46 (6 h, m) 1.95 (2H, m)

LC-MS rt 2.02 M+348

### Intermediate 7: 4-Bromo-1,2-diethoxy-benzene

To a well stirred mixture of diethoxybenzene (500 mg) and ammonium bromide (323 mg, 1.1 eq) in acetonitrile (20 ml) was added oxone (2.03 g, 1.1 eq). This suspension was stirred at rt for 4 h then the suspension filtered and the filtrate concentrated to give the title compound (723 mg, >90%). Used without further purification.

¹H(CDCl₃) 6.98 (2H, m), 6.73 (1H, d, J=8.85 Hz), 4.05 (4H, m), 1.44 (6H, m)

LC-MS rt 2.48 m/z 279 ES+

### Intermediate 8: 5-Bromo-2-methoxy-phenol

By the method of Meyers and Snyder, J. Org. Chem (1993), 58, 42, 5-amino-2-methoxyphenol (1 g) was suspended in sulfuric acid/MeOH./water (6 ml/3 ml/10 ml) and cooled to 0°. A solution of sodium nitrite (550 mg) in water (4.2 ml) added dropwise over 15 min, left to stir for 1 h at 0° then treated with cuprous bromide (583 mg) in water (10 ml) containing HBr (48%, 2 ml) over 15 min. After warming to rt for 1 h, the mixture was refluxed for 3 h and then cooled and extracted with diethyl ether. The combined extracts were dried and concentrated to a dark oil which was purified by column chromatography on Si with 0-15% EtOAc in petrol as eluant to give the desired compound as a clear oil (300 mg, 20%)

¹H(CDCl₃) 7.06 (1H, d, J=2.5 Hz), 6.96 (1H, dd, J 2.5, 8.2 Hz), 6.71 (1H, d, J=8.5 Hz), 5.63 (1H, s) 3.87 (3H, s)

LC-MS rt 2.27 m/z 201 & 203 ES+

### Intermediate 9: 4-Bromo-1,2-bis-trifluoromethoxy-benzene

This compound, as utilised in EP1013637, may be prepared by similar methods used to prepare intermediate 7 or intermediate 8. The starting material for these methods, 1,2-bis-trifluoromethoxy-benzene, may be prepared by alkylation of catechol with dibromodifluoromethane followed by conversion of the remaining bromosubstituents to fluoro by treatment with silver tetrafluoroborate or other source of fluoride ion.

### Intermediate 10: 4-Bromo-1,2-bis-difluoromethoxy-benzene

This compound may be prepared analogously to intermediate 8 from 3,4-Bis-difluoromethoxy-phenylamine (described in J. Pharm. Sci, 78, 7, 1989, 585) or by analogy with intermediate 7 from 1,2-bis-difluoromethoxy-benzene, itself prepared by alkylation/decarboxylation of catechol with ethyl chlorodifluoroacetate under base catalysis.

### Intermediate 11: 4-Bromo-2-ethoxy-1-methoxy-benzene

This known compound (Tercio J. et al Synthesis, 1987, 149-153) may be prepared by the alkylation of intermediate 8 with ethyl iodide under base catalysis eg NaH, DMF.

### Intermediate 12: 4-Bromo-1-ethoxy-2-methoxy-benzene

This known compound (Traverso G, Gazz. Chim. Ital, 1960, 778-791) may be prepared by the alkylation of 4-bromoguiiacol with ethyl iodide under base catalysis eg NaH, DMF.

### Example 1

### {6-[4-(2-Morpholin-4-yl-ethoxy)-phenyl]-quinazolin-4-yl}-(4-morpholin-4-yl-phenyl)-amine

### Step 1: 4-Amino-4′-(2-morpholin-4-yl-ethoxy)-biphenyl-3-carbonitrile

A mixture of intermediate 1 (0.5 g) 4-hydroxyphenylboronic acid (565 mg) and tetrakis (triphenylphosphine) palladium (0) (290 mg) in DME/2M aqueous sodium carbonate (2:1, 15 ml) was heated at reflux for 2 hr. The cooled mixture was diluted with ethyl acetate and washed with further aqueous base. The organic phase was dried (MgSO₄) and reduced onto silica gel. Flash chromatography with CH₂Cl₂ to 5% methanol in CH₂Cl₂ as eluant gave the coupling product, slightly contaminated with triphenylphosphine oxide by NMR. This material (340 mg) was heated with 2-chloroethyl morpholine hydrochloride (330 mg) and potassium carbonate (670 mg) in acetone (20 ml) at reflux overnight. The cooled reaction was partitioned between CH₂Cl₂ and 2M hydrochloric acid (aq). The acid phase was separated, basified and extracted with CH₂Cl₂ (2×). These organic washes were concentrated to give the title compound as a brown solid (342 mg)

LC-MS rt 2.15 M+324

¹H NMR δ 7.55 (2H, m), 7.38 (2H, d, J=8.21 Hz), 6.95 (2H, d, J=8.21 Hz), 6.78 (1H, m), 4.24 (2H, br s) 4.14 (2H, t, J=6.3 Hz), 3.72 (4H, m), 2.82 (2H, m), 2.59 (4H, m)

### Step 2: {6-[4-(2-Morpholin-4-yl-ethoxy)-phenyl]-quinazolin-4-yl}-(4-morpholin-4-yl-phenyl)-amine

A solution of the 4-Amino-4′-(2-morpholin-4-yl-ethoxy)-biphenyl-3-carbonitrile (330 mg) in DMF-DMA (1.2 ml) was heated at reflux for 1 h. The cooled mixture was concentrated in vacuo and the residue taken up in acetic acid (3 ml) and treated with 4-morpholinoaniline (180 mg). The mixture was heated to reflux for 2 h then allowed to cool and basified with 1M NaOH before being extracted into CH₂Cl₂ and dried (MgSO₄). Concentration in vacuo onto silica gel followed by chromatography with CH₂Cl₂/ethanol/ammonia (200:8:1) gave a solid which on trituration with diethyl ether and filtration, gave the title compound (214 mg)

¹H NMR δ 9.8 (1H, s), 8.75 (1H, s), 8.49 (1H, s), 8.12 (1H, d, J=8.85 Hz), 7.82 (3H, m), 7.64 (2H, d, J=8.2 Hz), 7.14 (2H, d, J=8.2 Hz), 7.02 (2H, d, J=8.2 Hz), 4.17 (2H, t, J=6.3 Hz), 3.75 (4H, m), 3.6 (4H, m), 3.11 (3H, m), 2.7 (2H, m)

LC-MS rt 3.03 M+512

### Example 2

### (4-Morpholin-4-yl-phenyl)-{6-[4-(3-morpholin-4-yl-propoxy)-phenyl]-quinazolin-4-yl}-amine

Step 1: A mixture of intermediate 3 (367 mg) and intermediate 6 (350 mg) with tetrakis(triphenylphosphine)palladium (0) (10%, 116 mg) in DME: 1M NaCO₃ aq. (2:1, ml), was heated to 80° for 12 h. The mixture was cooled, diluted with ethyl acetate and the phases separated. The organic phase was washed with water and dried before being absorbed onto silica gel and purified by SPE chromatography with CH₂Cl₂/EtOH/NH₃ (200:8:1) as eluant to give the coupled aniline as a brown oil (˜400 mg) LC-MS rt 2.03 m/z 334. This was dissolved in DMF-DMA (3 ml) and heated to 120° for 2 h. Cooled, concentrated and the residue purified by SPE chromatography on Si with CH₂Cl₂/EtOH/NH₃ (300:8:1 to 200:8:1) as eluant. This gave a pale gum that solidified on standing (213 mg, 63% over 2 steps) LC-MS rt 1.83 m/z 393. This pale solid (213 mg) was treated with 4-morpholinoaniline (97 mg) in AcOH (2 ml) and heated to 125° for 2 h. After concentration, the mixture was partitioned between DCM and aqueous sodium carbonate and the organic phases dried and concentrated to a solid which was triturated with diethyl ether/CH₂Cl₂/petrol to give, by filtration, the title compound (178 mg, 62%)

¹H NMR δ 9.79 (1H, s), 8.74 (1H, s), 8.48 (1H, s) 8.12 (1H, d, J=7.6 Hz), 7.8 (3H, m) 7.64 (2H, d, J=8.8 Hz), 7.09 (2H, d, J=8.8 Hz), 6.99 (2H, d, J=8.85 Hz), 4.09 (2H, m), 3.76 (4H, m), 3.58 (4H, m), 3.1 (4H, m), 2.38 (4H, m), 1.9 (2H, m)

LC-MS rt 1.96 m/z 524 ES−

### Example 3

### Preparation of [6-(3,4-Dimethoxy-phenyl)-quinazolin-4-yl]-(4-morpholin-4-yl-phenyl)-amine

### Step 1: 4-Amino-3′,4′-dimethoxy-biphenyl-3-carbonitrile

A mixture of 3,4-dimethoxyboronic acid (956 mg, 2 eq), intermediate 1 (640 mg, 1 eq), tetrakis (triphenylphosphine) palladium (0) (10%, 303 mg) in DME/2M aq sodium carbonate (2:1, 21 ml) was heated to 80° for 16 h.

The cooled reaction mixture was diluted with ethyl acetate and washed with further aq sodium carbonate then water. The dried organic phase was concentrated to a dark red gum which was dissolved in CH₂Cl₂ and loaded onto a SPE cartridge (Si, 20 g) and eluted with CH₂Cl₂. The major fractions containing product were combined and concentrated to a semi-solid which was triturated with diethyl ether and the desired compound isolated by filtration as a light brown solid (296 mg, 44%)

LC-MS rt 2.73 no ion observed

¹H (DMSO) 67.77 (1H, s), 7.71 (1H, d), 7.2 (1H, s), 7.17 (1H, d), 7.03 (1H, d), 6.91 (1H, d), 6.17 (2H, br s), 3.89 (3H, s), 3.83 (3H, s)

### Step 2: N′-(3-Cyano-3′,4′-dimethoxy-biphenyl-4-yl)-N,N-dimethyl-formamidine

A solution of aminobiphenyl (1,296 mg, 1.16 mmol) in DMF-DMA (excess, 1 ml) was heated to 100° for 1.5 h. The cooled reaction mixture was diluted with diethyl ether then petrol and the amidine product isolated by filtration to give, after drying, a light brown solid (313 mg, 87%)

¹H NMR (DMSO) δ 7.99 (1H, s) 7.91 (1H, d, J=1.9 Hz), 7.79 (1H, dd, J 8.2, 1.9 Hz), 7.2 (3H, m), 6.99 (1H, d, J=8.2 Hz), 3.84 (3H, s), 3.78 (3H, s), 3.08 (3H, s) 3.01 (3H, s)

LC-MS rt 2.31 m/z 309.95

### Step 3: [6-(3,4-Dimethoxy-phenyl)-quinazolin-4-yl]-(4-morpholin-4-yl-phenyl)-amine

The amidine (II, 112 mg) and 4-morpholinoaniline (1 eq, 65 mg, light brown solid ex Lancaster 98%+) in acetic acid (0.5 ml) were heated to 120° for 1 h. The cooled reaction mixture was basified with NaOH (2N aq) and the resulting yellow solid isolated by filtration and dried in vacuo.

This gave the title compound as a yellow solid (130 mg, 80%)

¹H NMR (DMSO) δ 9.78 (1H, s), 8.72 (1H, s), 8.48 (1H, s), 8.16 (1H, d, J 9.5 Hz), 7.78 (1H, d, J8.2 Hz), 7.64 (2H, d, J 8.8 Hz ), 7.42 (2H, m), 7.12 (1H, d, J 8.8 Hz ), 7.0 (2H, d, J9.5 Hz), 3.90 (3H, s), 3.83 (3H, s), 3.76 (4H, m), 3.1 (4H, m)

LC-MS rt 2.47 m/z 443

### Example 4

### Preparation of 6-(3,4-Diethoxy-phenyl)-quinazolin-4-yl]-(4-morpholin-4-yl-phenyl)-amine

A mixture of intermediate 7 (200 mg) and intermediate 5 (490 mg) were combined with terakis(triphenylphosphine) palladium (0) (95 mg0 in DME (3 ml) and sodium carbonate (1 ml) and heated to 100° overnight. The cooled mixture was diluted with water and extracted into CH₂Cl₂. The organic phases were combined, concentrated and partially purified by column chromatography with CH₂Cl₂/EtOH/NH₃ (200:8:1) to give material (290 mg) that was heated in acetic acid (3 ml) with 4-morpholinoaniline (230 mg) at 80°over 4 h. The mixture was diluted with water, basified with 2N NaOH and the resulting precipitate isolated by filtration and washed with water and diethyl ether, dried then washed with ethyl acetate and pet ether and re-dried to give the title compound as a yellow solid (215 mg, 70%).

¹H NMR (DMSO) δ 9.76 (1H, s), 8.7 (1H, s), 8.48 (1H, s), 8.16 (1H, d, J 8.85 Hz), 7.78 (1H, d, J 8.85 Hz), 7.64 (2H, d, J 8.85 Hz), 7.42 (2H, m), 7.11 (1H, d, J 8.2 Hz), 6.99 (2H, d, J 8.85 Hz), 4.13 (4H, m), 3.76 (4H, m), 3.11 (4H, m), 1.36 (6H, m)

LC-MS rt 2.40 m/z 471 ES+

### Example 5

### Preparation of {6-[3-Fluoro-4-(2-morpholin-4-yl-ethoxy)-phenyl]-quinazoline-4-yl}-(4-morpholin-4-yl-phenyl)-amine

A mixture of intermediate 1 (100 mg), 4-hydroxy-3-fluorophenyl boronic acid (128 mg) and tetrakis(triphenylphosphine)palladium (0) (47 mg) in DME (3 ml) and aq 2M sodium carbonate (1 ml) were heated for 80° for 3 h. The cooled mixture was diluted with ethyl acetate and washed with water. The organic phase was dried, concentrated and purified by column chromatography with CH₂Cl₂/EtOH/NH₃ (300:8:1) to give the desired biphenyl compound (70 mg, LC-MS rt 2.29 m/z 227 ES−) which was dissolved in acetone (2 ml) and treated with chloroethyhnorpholine hydrochloride (63 mg) and potassium carbonate (128 mg) and heated to reflux for 16 h. After concentration, the residue was taken up in CH₂Cl₂ and washed with water. The organic phase was dried and concentrated to a brown solid (79 mg) which was partially purified by chromatography on silica gel with CH₂Cl₂/EtOH/NH₃ (300:8:1) to give material (69 mg, LC-MS rt 1.95 m/z 342 ES+) which was heated in DMF-DMA (1 ml) for 2 h at 80°. The solvent was removed in vacuo and the residue (74 mg, LC-MS rt 1.85 m/z 397 ES+) heated with 4-morpholinoaniline (68 mg) in acetic acid (1 ml) at 80° for 4 h. The cooled mixture was diluted with water and basified with 2M NaOH before being extracted with ethyl acetate. The combined organics were dried and concentrated to a dark solid that was purified by chromatography on silica with CH₂Cl₂/EtOH/NH₃ (300:8:1 to 100:8:1) as eluant. This gave the title compound (31.5 mg, 31%)

¹H NMR (DMSO) δ 9.79 (1H, s), 8.76 (1H, s), 8.15 (1H, d, J 8.5 Hz), 7.8 (2H, m), 7.66 (3H, m), 7.35 (1H, t, J 8.85 Hz), 6.99 (2H, d, J 8.85 Hz), 4.25 (2H, t, J 5.7 Hz), 3.76 (4H, m), 3.59 (4H, m), 3.11 (4H, m), 2.75 (2H, t, J 5.69 Hz)

LC-MS rt 2.01 m/z 530 ES+

### Example 6

### 2-Methoxy-5-[4-(4-morpholin-4-yl-phenylamino)-quinazolin-6-yl]-phenol

A mixture of intermediate 8 (300 mg), intermediate 5 (659 mg) and tetrakis(triphenylphosphine)palladium (0) (170 mg) in DME (5 ml) and aq 2M sodium carbonate (1 ml) were heated for 80° for 16 h. The cooled mixture was diluted with water and extracted with CH₂Cl₂. The organic phase was dried and concentrated onto silica to give, after chromatography with CH₂Cl₂/EtOH/NH₃ (600:8:1 to 300:8:1) as eluant, a slightly impure sample of the amidine (190 mg LC-MS rt 1.94 m/z 296 ES+) which was heated with 4-morpholinoaniline (171 mg) in acetic acid (2 ml) at 80° for 3 h. On cooling, the mixture was diluted with water, basified with 2M NaOH and extracted into CH₂Cl₂. The organic phase was dried and concentrated onto silica gel. Purification by column chromatography gave material of 90% purity so a sample of this material (90 mg) was further purified by prep HPLC to give the title compound.

¹H NMR (DMSO) δ 9.88 (1H, s), 9.2 (1H, br s), 8.76 (1H, s), 8.53 (1H, s), 8.11 (1H, d), 7.82 (1H, d), 7.71 (2H, d), 7.37 (2H, m), 7.13 (1H, d), 7.04 (2H, d), 3.9 (3H, s); 3.82 (4H, m), 3.17 (4H, m).

LC-MS rt 2.16 m/z 429 ES+

### Example 7

### [6-(3,4-Bis-trifluoromethoxy-phenyl)-quinazolin-4-yl]-(4-morpholin-4-yl-phenyl)-amine

This compound may be prepared by the method of Example 4 using Intermediate 9 and intermediate 5.

### Example 8

### [6-(3,4-Bis-difluoromethoxy-phenyl)-quinazolin-4-yl]-(4-morpholin-4-yl-phenyl)-amine

This compound may be prepared by the method of Example 4 using Intermediate 10 and intermediate 5.

### Example 9

### {6-[4-Methoxy-3-(2-morpholin-4-yl-ethoxy)-phenyl]-quinazolin-4-yl}-(4-morpholin-4-yl-phenyl)-amine

Example 6 (100 mg), chloroethylmorpholine hydrochloride (48 mg) and potassium carbonate (95 mg) in DMF (2 ml) were heated to 100° for 16 h. Cooled, filtered and the filter cake washed through with CH₂Cl₂. The filtrate was washed with water, dried and concentrated onto silica before being partially purified by chromatography with CH₂Cl₂/EtOH/NH₃ (600:8:1 to 200:8:1) as eluant. The fraction containing product was further purified by prep HPLC to give an orange gum (54 mg) that on trituration with ethyl acetate yielded the title compound (9 mg).

¹H NMR (DMSO) δ 9.8 (1H, s), 8.72 (1H, s), 8.49 (1H, s), 8.16 (1H, d, J 8.2 Hz), 7.78 (1H, d, J 8.85 Hz), 7.64 (2H, d, J 8.85 Hz), 7.45 (2H, m), 7.12 (1H, d, J 8.2 Hz), 6.99 (2H, d, J 8.85 Hz), 4.23 (2H, m), 3.83 (3H, s), 3.76 (4H, m), 3.57 (4H, m), 3.1 (4 h, m), 2.74 (2H, m)

LC-MS rt 1.91 m/z 542 ES+

### Example 10

### {6-[3-Methoxy-4-(2-morpholin-4-yl-ethoxy)-phenyl]-quinazolin-4-yl}-(4-morpholin-4-yl-phenyl)-amine

4-bromoguaiacol (7 g) and chloroethylmorpholine hydrochloride (7.06 g) in acetone (100 ml) was treated with potassium carbonate (14.27 g) and refluxed for 16 h. After filtration, the filtrate was concentrated onto silica gel and purified by column chromatography with petrol to 20% ethyl acetate in petrol as eluant to give the alkylated product as a clear oil. A portion of this material (1 g), intermediate 5 (1.42 g) and tetrakis(triphenylphosphine)palladium (0) (365 mg) in DME (10 ml) and aq 2M sodium carbonate (3 ml) were heated for 80° for 16 h. The mixture was diluted with water and extracted into CH₂Cl₂. The combined organic extracts were dried, concentrated onto silica and purified by chromatography with CH₂Cl₂/EtOH/NH₃ (600:8:1 to 200:8:1) as eluant. This gave the desired coupled product in a single fraction (900 mg, 61%). A portion of this material (100 mg) was treated with 4-morpholinoaniline (68 mg) in acetic acid (1 ml) at 80° for 3 h. On cooling, the mixture was diluted with water, basified with 2M NaOH and extracted into CH₂Cl₂. The organic phase was dried and concentrated onto silica gel. Purification by column chromatography gave the title compound (20 mg)

¹H NMR (DMSO) δ 9.78 (1H, s), 8.72 (1H, s), 8.48 (1H, s), 8.16 (1H, d, J 8.85 Hz), 7.78 (1H, d, J 8.85 Hz)m, 7.64 (2H, d, J 8.85 Hz), 7.43 (2H, m), 7.14 (1H, d, J 8.85 Hz), 7.0 92H, d, J 8.85 Hz), 4.14 (2H, m), 3.9 (3H, s), 3.76 (4H, m), 3.59 (4H, m), 3.1 (4H, m), 2.72 (2H, m)

LC-MS rt 1.91 m/z 542 ES+

### Example 11

### {6-[3-Fluoro-4-(3-morpholin-4-yl-propoxy)-phenyl]-quinazolin-4-yl}-(4-morpholin-4-yl-phenyl)-amine

### Step 1: 2-Fluoro-4-[4-(4-morpholin-4-yl-phenylamino)-quinazolin-6-yl]-phenol

Coupling of 3-fluoro-4-hydroxy boronic acid (600 mg) and intermediate 2 (767 mg) under catalysis by tetrakis(triphenylphosphine)palladium (0) (300 mg) in DME (18 ml) and aq 2M sodium carbonate (9 ml) were heated at reflux for 16 h. The cooled reaction mixture was acidified with 2M HCl and the aqueous decanted away from the resulting tacky gum. This material was azeotroped with toluene then treated with 4-morpholinoaniline (479 mg) in acetic acid (10 ml) at reflux for 1.5 h. The cooled mixtures were concentrated, diluted with water and basified with aq. sodium bicarbonate. The aqueous was decanted and acetonitrile added with stirring to the residue to give a suspension. Filtration gave the desired quinazoline as a dark green solid (540 mg, 51%).

¹H NMR (DMSO) δ 10.2 (1H, s), 9.13 (1H, s), 8.89 (1H, s), 8.53 (1H, d), 8.06 (6H, m), 7.4 (4H, m), 4.18 (4H, m), 3.54 (4H, m)

LC-MS rt m/z ES+

### Step 2: {6-[3-Fluoro-4-(3-morpholin-4-yl-propoxy)-phenyl]-quinazolin-4-yl}-(4-morpholin-4-yl-phenyl)-amine

Material from step 1 (50 mg) was treated with 3-chloropropylmorpholine hydrochloride (36 mg) and potassium carbonate (75 mg) in DMF (2 ml) at 70°. The cooled reaction mixture was cooled and concentrated then suspended in water and filtered. The solid isolated was purified by chromatography on silica with CH₂Cl₂/EtOH/NH₃ (100:8:1) as eluant to give the title compound.

¹H NMR (DMSO) 9.8 (1H, s), 8.77 (1H, s), 8.50 (1H, s), 8.15 (1H, d, J 8.85 Hz), 7.8 (2H, m), 7.63 (3H, m), 7.33 (1H, t, J 8.85 Hz), 7.03 (2H, d, J 8.85 Hz), 4.17 (2H, m), 3.76 (4H, m), 3.58 (4H, m), 3.1 (4H, m), 2.38 (3H, m), 1.93 (2H, m)

LC-MS rt m/z ES+

### Example 12

### [6-(3-Ethoxy-4-methoxy-phenyl)-quinazolin-4-yl]-(4-morpholin-4-yl-phenyl)-amine

This compound may be prepared by reaction of intermediate 11 with intermediate 5 by a similar procedure to the preparation of example 4.

### Example 13

### [6-(4-Ethoxy-3-methoxy-phenyl)-quinazolin-4-yl]-(4-morpholin-4-yl-phenyl)-amine

This compound may be prepared by reaction of intermediate 12 with intermediate 5 by a similar procedure to the preparation of example 4.

### Activity Example

### Cells Used:

HCV replicon cells Huh 9B (ReBlikon), containing the firefly luciferase—ubiquitin—neomycin phosphotransferase fusion protein and EMCV-IRES driven HCV polyprotein with cell culture adaptive mutations.

### Cell Culture Conditions:

Cells were cultured at 37° C. in a 5% CO₂ environment and split twice a week on seeding at 2×10E6 cells/flask on day 1 and 1×10E6 3 days later. Some 0.25 mg/ml G418 was added to the culture medium (125 ul per 25 ml) but not the assay medium.

The culture medium consisted of DMEM with 4500 g/l glucose and glutamax (Gibco 61965-026) supplemented with 1× non-essential amino acids, penicillin (100 IU/ml)/streptomycin (100 μg/ml), FCS (10%, 50 ml) and 1 mg/ml G418 (Invitrogen cat no 10131-027) & 10% foetal calf serum.

### Assay Procedure:

A flask of cells was trypsinised and a cell count carried out. Cells were diluted to 100,000 cells/ml and 100 μl of this used to seed one opaque white 96-well plate (for the replicon assay) and one flat-bottomed clear plate (for the tox assay) for every seven compounds to be tested for IC50. Wells G12 and H12 were left empty in the clear plate as the blank. Plates were then incubated at 37° C. in a 5% CO₂ environment for 24 h.

On the following day compound dilutions are made up in medium at twice their desired final concentration in a clear round bottomed plate. All dilutions have a final DMSO concentration of 1%.

Once the dilution plate had been made up, controls and compounds were transferred to the assay plate (containing the cells) at 100 μl /well in duplicate plates.

Exception: in the white (replicon) plate, no compound was added to wells A1 and A2 and 100 μl of 1% DMSO was added to these instead. In the clear (Tox) plate, wells E12 & F12 only contained the DMSO control. Plates were then incubated at 37° C. with 5% CO₂ for 72 h.

At the end of the incubation time, the cells in the white plate were harvested by washing with 200 μl/well of warn (37° C.) PBS and lysed with 20 μl cell culture lysis buffer (Promega). After 5 min incubation @ RT, luciferin solution was added to the luciferase assay buffer (LARB at 200 μl per 10 ml LARB. The M injector of the microplate luminometer (Lmax, Molecular Devices) was primed with 4×300 l injections. Plate were inserted into the luminometer and 100 μl luciferase assay reagent was added by the injector on the luminometer. The signal was measured using a 1 second delay followed by a 4 second measurement programme. The IC50, the concentration of the drug required for reducing the replicon level by 50% in relation to the untreated cell control value, can be calculated from the plot of the percentage reduction of the luciferase activity vs. drug concentration.

The clear plate was stained with 100 μl 0.5% methylene blue in 50% ethanol at RT for 1 h, followed by solvation of the absorbed methylene blue in 100 μl per well of 1% lauroylsarcosine. Absorbance of the plate was measured on a microplate spectrophotometer (Molecular Devices) and the absorbance for each concentration of compound expressed as a proportion of the relative DMSO control. The TD50, the concentration of drug required to reduce the total cell area by 50% relative to the DMSO controls can be calculated by plotting the absorbance at 620 nm vs drug concentration.

TABLE 1

| | Replicon IC50 (<1 uM = ***; | Replicon TD5O (>25 uM = ***; |
|---|---|---|
| Exam- | <5 uM = **; <25 uM = *) | >10 uM = **; >1 uM = *) |
| ple no | uM | uM |
| 1 | ** | *** |
| 2 | *** | ** |
| 3 | *** | ** |
| 4 | *** | ** |
| 5 | *** | *** |

1. A compound which is a quinazoline derivative of formula (Ia), or a pharmaceutically acceptable salt thereof, 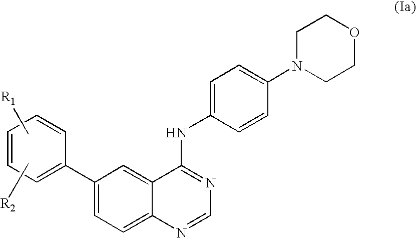
wherein R₁ and R₂ are the same or different and represent hydrogen, halogen, -L-O—R₃, -L-O-L-A or -L-O-L′-A′, wherein
each L is the same or different and represents a direct bond or a C₁-C₄ alkylene group;
L′ represents a direct bond or a C₂-C₄ alkylene group;
R₃ represents hydrogen, C₁-C₄ alkyl or C₁-C₄ haloalkyl;
A represents a 5- to 10-membered heterocyclyl group; and
A′ represents a C₆-C₁₀ aryl group;
wherein at least one of R₁ and R₂ is -L-O—R₃, -L-O-L-A or -L-O-L′-A′.

2. A compound according to claim 1, wherein R₁ is located para to the quinazoline ring and R₂ is located meta to the quinazoline ring.

3. A compound according to claim 1, wherein R₁ represents -L-O—R₃, -L-O-L-A or -L-O-L′-A′, and R₂ represents hydrogen, halogen -L-O—R₃, -L-O-L-A or -L-O-L′-A′, provided that when R₁ represents -L-O-L-A or -L-O-L′-A′, R₂ represents hydrogen, halogen or -L-O—R₃.

4. A compound according to claim 1, wherein when R₁ or R₂ represents -L-O—R₃, the group L is a direct bond or C₁-C₂ alkylene, and R₃ is hydrogen, C₁-C₂ alkyl or C₁-C₂ haloalkyl.

5. A compound according to claim 1, wherein the group -L-O-L-A is a group —O-L-A or —(C₁-C₂ alkylene)-O-L-A, wherein L is a direct bond or a C₁-C₄ alkylene group.

6. A compound according to claim 1, wherein A is a morpholinyl group.

7. A compound according to claim 1, wherein the group -L-O-L′-A′ is a group —O-L′-A′ or —(C₁-C₂ alkylene)-O-L′-A′, wherein L′ is a direct bond or a C₂-C₄ alkylene group.

8. A compound according to claim 1, wherein A′ is a phenyl group.

9. A compound according to claim 1, wherein the quinazoline derivative of formula (Ia) is a quinazoline derivative of formula (I), 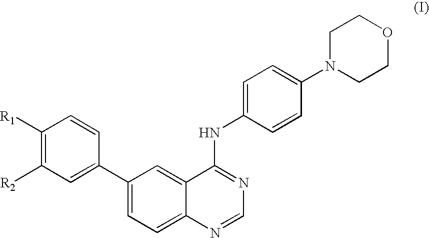
wherein R₁ and R₂ are the same or different and represent hydrogen, halogen, —O—R₃, —O-L-A or —O-L′-A′, wherein
L represents a C₁-C₄ alkylene group;
L′ represents a C₂-C₄ alkylene group;
R₃ represents hydrogen, C₁-C₂ alkyl or C₁-C₂ haloalkyl;
A represents morpholinyl; and
A′ represents phenyl;
wherein at least one of R₁ and R₂ is —O—R₃, —O-L-A or —O-L′-A′.

10. A compound according to claim 9, wherein R₁ and R₂ are the same or different and represent hydrogen, halogen, —O—R³ or —O-L-A, wherein
L represents a C₁-C₄ alkylene group;
R₃ represents hydrogen, C₁-C₂ alkyl or C₁-C₂ haloalkyl; and
A represents a morpholinyl group,
wherein at least one of R₁ and R₂ represents —O—R₃ or —O-L-A.

11. (canceled)

12. A pharmaceutical composition which comprises a quinazoline derivative of claim 1, or a pharmaceutically acceptable salt thereof, and a pharmaceutically acceptable carrier or diluent.

13-21. (canceled)

22. A product containing:
(a) a quinazoline derivative of claim 1, or a pharmaceutically acceptable salt thereof; and
(b) interferon, or an interferon derivative and/or ribavirin or a ribavirin derivative;
for simultaneous, separate or sequential use in the treatment of the human or animal body.

23. A method of treating a patient suffering from or susceptible to a flaviviridae infection, which method comprises administering to said patient an effective amount of a quinazoline derivative of claim 1 or a pharmaceutically acceptable salt thereof.

24. A method according to claim 23, wherein the flaviviridae infection is a pestivirus infection.

25. A method according to claim 24, wherein the pestivirus infection is an infection by a bovine viral diarrhea virus, classical swine fever virus or border disease virus.

26. A method according to claim 23, wherein the flaviviridae infection is a flavivirus infection.

27. A method according to claim 26, wherein the flavivirus infection is an infection by a yellow fever virus, dengue fever virus, Japanese encephalitis virus or tick borne encephalitis virus.

28. A method according to claim 23, wherein the flaviviridae infection is a hepacivirus infection.

29. A method according to claim 28, wherein the hepacivirus infection is an infection by a hepatitis C virus.

30. A method according to claim 29, wherein the medicament further comprises (a) interferon or a derivative thereof and/or (b) ribavirin or a derivative thereof.

31. A method according to claim 30 wherein the interferon derivative is PEG-interferon and/or the ribavirin derivative is viramidine.
